(19) United States
(12) Patent Application Publication
(10) Pub. No.: US 2016/0193251 A1
(43) Pub. Date: Jul. 7, 2016

(54) METHOD FOR TREATING ALS VIA THE INCREASED PRODUCTION OF FACTOR H

(71) Applicant: BioViva USA Inc., Bainbridge Island, WA (US)

(72) Inventor: Jason Williams, Gulf Shores, AL (US)

(21) Appl. No.: 15/043,499

(22) Filed: Feb. 12, 2016

Related U.S. Application Data
(63) Continuation of application No. 14/181,405, filed on Feb. 14, 2014, now abandoned.
(60) Provisional application No. 61/765,334, filed on Feb. 15, 2013.

(51) Int. Cl. A61K 35/28; A61K 38/51; A61K 38/17; C12N 5/0775; A61K 38/30

(52) U.S. Cl. CPC A61K 35/28; C12N 5/0667; A61K 38/30; A61K 38/1709; A61K 38/51; C12N 2510/00; C12N 2501/70; A61K 48/00

(57) ABSTRACT

Methods and systems for the treatment for ALS incorporating stem cells harvested from the subject to be treated. These stem cells may be genetically altered with the addition of several genes of interest. Then, the patient will receive systemic gene therapy for the muscles and directed specifically at motor neurons. In this multi-pronged treatment approach, the stem cells provide immune regulation and the regeneration of motor neurons. And, the new motor neurons carry the added genes, which are protective against motor neuron death from ALS. The systemic therapy increases the amount of genes, which further reduces the effects of ALS. Additional gene therapy administered in the muscle will be further protective of the axon, while maintaining muscle mass and function.

### CROSS REFERENCE TO RELATED APPLICATIONS

This application claims benefit of priority to U.S. provisional patent application Ser. No. 61/765,334 filed Feb. 15, 2013; the content of which is herein incorporated by reference in its entirety.

### FIELD OF THE INVENTION

The present invention relates generally to a method of treating neurodegenerative related disorders, and more specifically to a stem cell, gene therapy or a combined stem cell-gene therapy treatment approach for treating Amyelotrophic Lateral Sclerosis, Multiple Sclerosis, Parkinson's Disease and/or Alzheimer's Disease.

### BACKGROUND OF THE INVENTION

Amyelotrophic lateral sclerosis (ALS), also known as Lou Gehrig's disease in the United States, is a fatal motor neuron disease (MND) with adult onset and relatively short course, culminating in death within three to five years post-diagnosis. This neurodegenerative disease is characterized mainly by the progressive degeneration of upper and lower motor neurons (MNs) in the spinal cord, brainstem, and motor cortex. As MNs degenerate, muscles lose strength, and voluntary movements are compromised. Death is usually caused by respiratory failure, when diaphragm and intercostal muscles become disabled.

In the United States, the prevalence of ALS is approximately 30,000, and the incidence is slightly greater (60%) in the male population. The disease generally occurs between the ages of 40 and 70 years.

Amyotrophic lateral sclerosis (ALS) is a neurodegenerative disease with unknown and poorly understood etiology.

Although clinically indistinguishable, ALS can occur in one of two forms; a most common or sporadic (sALS) than, which affects approximately 90% of the patients, or a familial (fALS) form linked to specific genetic mutations, which affects approximately 10% of ALS patients.

Whether sporadic or caused by specific genetic mutations, the disease invariably has a common pathological feature: the selective death of MNs. Oxidative stress, neurofilament abnormalities, excitotoxicity, apoptosis, mitochondrial dysfunction, defective axonal transport, mutations in RNA binding proteins, and inflammation are among the multiple factors playing a role in the pathogenesis of ALS.

Attempts at successfully curing, slowing the progression, or ameliorating the symptoms have been met with very minimal success, therefore there is a very pressing need to find ways to combat the disease and its progression and underlying symptoms. The present invention has evaluated the complexity of the disease and developed a multi-prong treatment approach and/or a personalized medicine approach to attacking the disease state through various physiological pathways.

### SUMMARY OF THE INVENTION

The present invention provides methods and systems for preventing, treating and/or ameliorating the symptoms of neurodegenerative disorders, and more specifically to preventing, treating and/or ameliorating the symptoms associated with ALS through the use of gene therapy, stem cell therapy, or a combination thereof.

In one aspect of the invention, a method of increasing the presence of Factor H in a mammalian subject is provided, which includes harvesting adipose tissue from the subject; purifying stem cells from the adipose tissue; treating the stem cells with a compound that increases secretion of Factor H, optionally Selegeline; and introducing the treated stem cells into the subject. Using this approach, the increased factor H inhibits the effect of complement on neuronal cells.

In related embodiments, the invention also includes methods of treating a motor neuron disease, which includes harvesting stem cells from a patient with the motor neuron disease; genetically altering the stem cells by the addition of one or more genes selected from the group consisting of IGF-1, TDP-42, and factor H; administering the genetically altered stem cells systemically to the patient; wherein the systemic administration serves to carry added genes which are protective against motor neuron death, and which further increases the amount of selective genes which further reduce the effects of motor neuron disease; and optionally administering additional selected gene therapy components intramuscularly, wherein the intramuscular administration serves to protect the axon and assist with maintaining muscle mass and function.

In related embodiments the present invention applies gene therapy or stem cell therapy alone, or combined together, where the stem cell therapy includes neural reprogrammed stem cells. In still further embodiments the methods use adipose derived stem cells which have undergone reprogramming using the monoamine oxidase inhibitor Selegeline. Selegeline activates the gene Oct4, which results in reprogramming the adipose derived stem cells into motor neurons. Still further embodiments may combine genetically engineered stem cells along with neural and systemic cell therapy to result in significantly improved treatment outcomes in patients with ALS.

### BRIEF DESCRIPTION OF THE DRAWINGS

The invention will be better understood and objects other than those set forth above will become apparent when consideration is given to the following detailed description thereof. Those of skill in the art will understand that the drawings, described below, are for illustrative purposes only. The drawings are not intended to limit the scope of the present teachings in any way. Such description makes reference to the annexed drawing wherein:

FIG. 1 is an illustration which demonstrates the manner in which complement binds to the neuron cell membrane.

FIG. 2 is an illustration which demonstrates how mesenchymal cells produce Factor H, which inhibits Complement by removing it from the cell membrane of the neuron.

FIG. 3 is an illustration which demonstrates the production of Factor H by stem cells by inhibiting the attack of Complement, which is one of the major factors causing nerve destruction in ALS.

FIG. 4 is an illustration which demonstrates Precision Stem Cell's PRCN 829 AAV gene therapy approach for delivering multiple genes, which increase production of Factor H and multiple neural growth factors. This combination of genes inhibits the destruction of neurons in ALS.

FIG. 5 is an illustration which demonstrates Precision Stem Cell's PRCN 829 gene therapy introducing multiple genes into the stem cells, which increase their production of Factor H, and multiple neural growth factors.

FIG. 6 is an illustration which demonstrates Factor H regulating Complement, which keeps it from attacking the patient's own cells. Factor H removes Complement from the neuron's cell membrane.

### DETAILED DESCRIPTION OF THE PREFERRED EMBODIMENTS

Often successful treatments in medicine require a combination of therapies to prove effective. Embodiments of the present invention for effective treatments in ALS may comprise of two, three or more different therapies combined to obtain a synergistic effect. The hallmark of ALS is death of motor neurons (MN). The etiology is poorly understood, but seems to result from a cascade of genetic and immune abnormalities, which will ameliorate the end outcome of motor neuron death.

Embodiments of the present invention include performing adult adipose derived stem cell therapy to treat patients with ALS. Amyelotrophic Lateral Sclerosis (ALS) is a fatal disease with no treatment options. Since ALS is a neurological disorder, it was believed that patients would need neural stem cells, not the mesenchymal type stem cells that we have obtained from harvested adipose tissue. But, because neural stem cells are difficult to attain for various physiological and regulatory reasons, a new technical approach that targets the conversion or reprogramming of mesenchymal type stem cells that are obtained from adipose tissue recovered from the same patient to be treated has been generated. In addition, it has been further found that treating the harvested stem cells with a drug called Selegeline was able to reprogram the stem cells from fat into neural like cells.

These reprogrammed stem cells are found to produce a substance which slows or inhibits the underlying disease process. In addition, it is believed that there will be a similar result for MS, Parkinson's and Alzheimers.

ALS may be considered a misguided attack from the complement component of the immune system. There are studies that show inhibiting complement in mouse models reduces the disease. This may explain why the stem cell therapy approach is working, namely, it inhibits complement by secreting a complement inhibitor, Factor H.

A technical approach of the present invention is to expand on this finding by harvesting and purifying stem cells from a subject suffering from ALS, MS, Parkinson's or Alzheimers and treat them with a gene therapy approach to boost and extend the effect of inhibiting complement, which in some embodiments is accomplished by increasing the secretion of Factor H, which results in complement inhibition. The gene therapy may utilize Adeno-associated virus (AAV) and adenoviral vectors. An AAV approach is most preferred for gene therapy. In these embodiments, the methods may include loading or packing the gene for Factor H in the AAV for delivery, then treating or transfecting stem cells to increase production and secretion of Factor H. Using this approach, increasing the amount of Factor H further inhibits or reduces the underlying disease process.

The skilled artisan will appreciate that the invention further advances an approach to medical treatment referred to as “personalized medicine.” This means that therapies are not generated by large scale pharmaceutical manufacturing processes to produce a plurality of identical therapeutics, but instead are individual and “tailor maid” for each patient. To this end, the embodiments for treatment methods for ALS preferably include the use of stem cells harvested from the same patient that is to be treated for ALS. These stem cells are then genetically altered with the addition of nucleic acid sequences that that when transfected into the cells are able to produce a beneficial effect, such as the production of polypeptides for secretion into the surrounding biological environment. In addition or alternative embodiments, the patient may receive systemic gene therapy for the muscles and directed specifically at motor neurons. In this multi-pronged treatment approach, the stem cells provide immune regulation and the regeneration of motor neurons. These new motor neurons may carry the added genes, which are protective against motor neuron death from ALS. The systemic therapy increases the amount of genes, which further reduces the effects of ALS. Also, gene therapy treatments in the muscle are contemplated embodiments that may be protective of the axon, while maintaining muscle mass and function.

Targeting Complement for the Treatment of ALS

Complement (C3 and C5) seem to be the main cause in ALS. It is this misguided attack that starts the cascade of ALS with motor neuron damage and ultimately death. The key regulator in this process is called Factor H. Factor H keeps Complement from attacking self. Mesenchymal stem cells are shown to produce Factor H. This inhibits Complement and generally results in some improvement of ALS symptoms. The Factor H produced by stem cells helps the damaged or stunned neurons to begin functioning again. Most people believe that the main theory behind stem cells is that they regenerate nerves. They do to some extent, but the newly regenerated nerves will be attacked by the same process that causes ALS (attack from Complement). So, one technical approach of the invention is to stop the underlying cause first. While normal stem cells will make Factor H, they only do so to a small level. Therefore, some of the technical achievements is the increased production of Factor H and maintaining its production over time. In some embodiments, Factor H is increased ten-fold or more. In other embodiments, Factor H is increased 100 fold or more. In other embodiments, Factor H is increased one thousand fold or more. Another challenge with using untreated stem cells tend to differentiate or die and thus the production of Factor H will fade. Accordingly, by enhancing or maintaining Factor H production, the methods of the present invention can provide a treatment that is prolonged. It is estimated that the effect may last many years

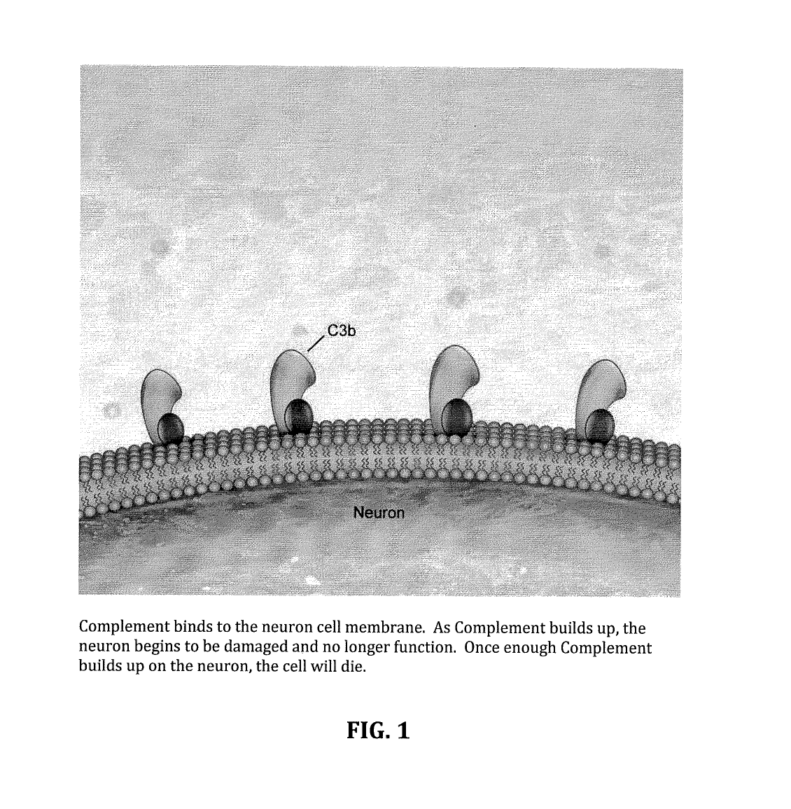

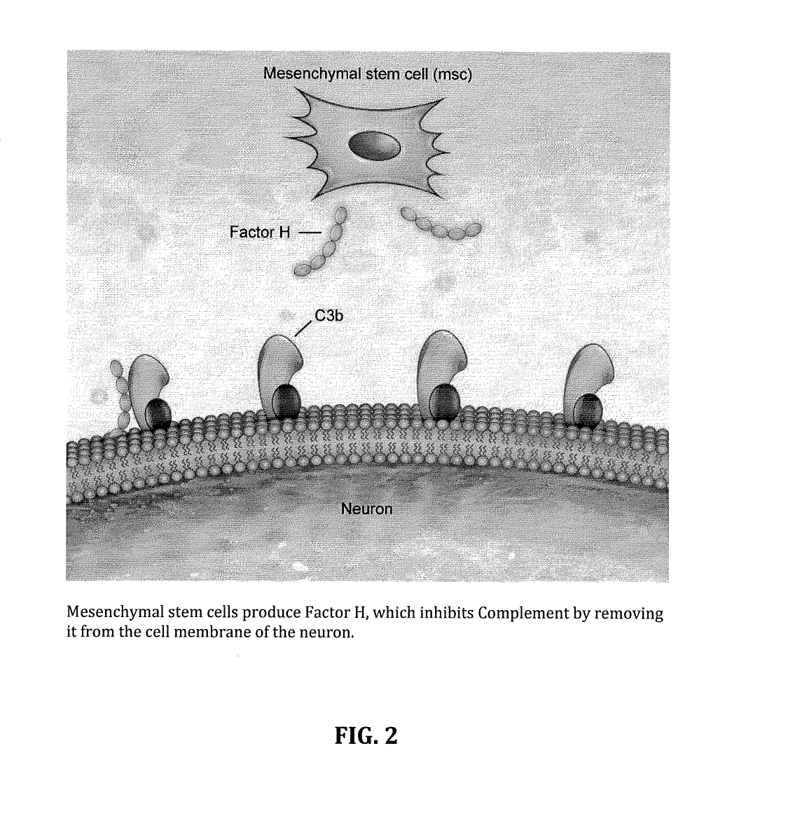

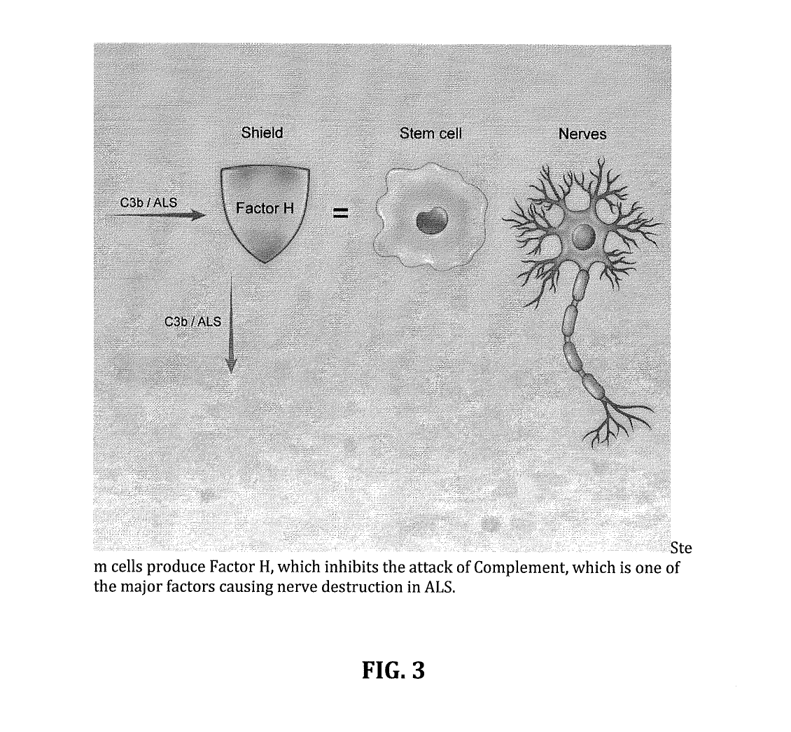

An example of the Complement process and treatment with stem cells is shown in FIGS. 1-3. In FIG. 1 Complement (C3b) is shown binding to the neuron cell membrane. As Complement builds up, the neuron begins to be damaged and no longer functions. Once enough Complement builds up on the neuron, the cell dies. In FIG. 2, the illustration shows the same neuron cell membrane in the presence of a mesenchymal stem cell (msc). Mesenchymal stem cells produce Factor H, which inhibits Complement by removing it from the cell membrane of the neuron. As shown in FIG. 3 the stem cells produce Factor H which serve as a shield for the nerves because Factor H inhibits the attack of Complement, which is one of the major factors causing nerve destruction in ALS.

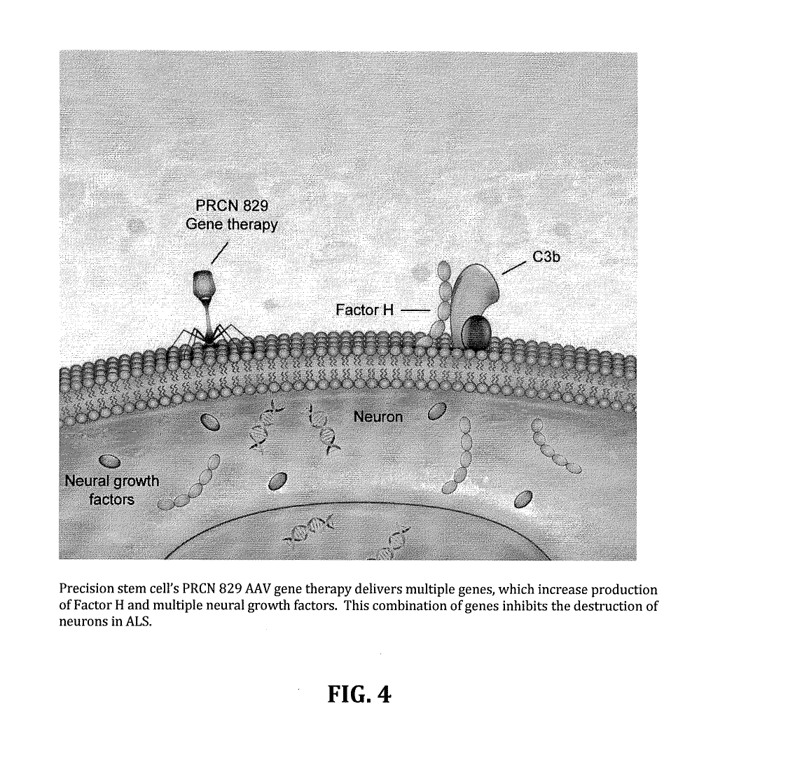

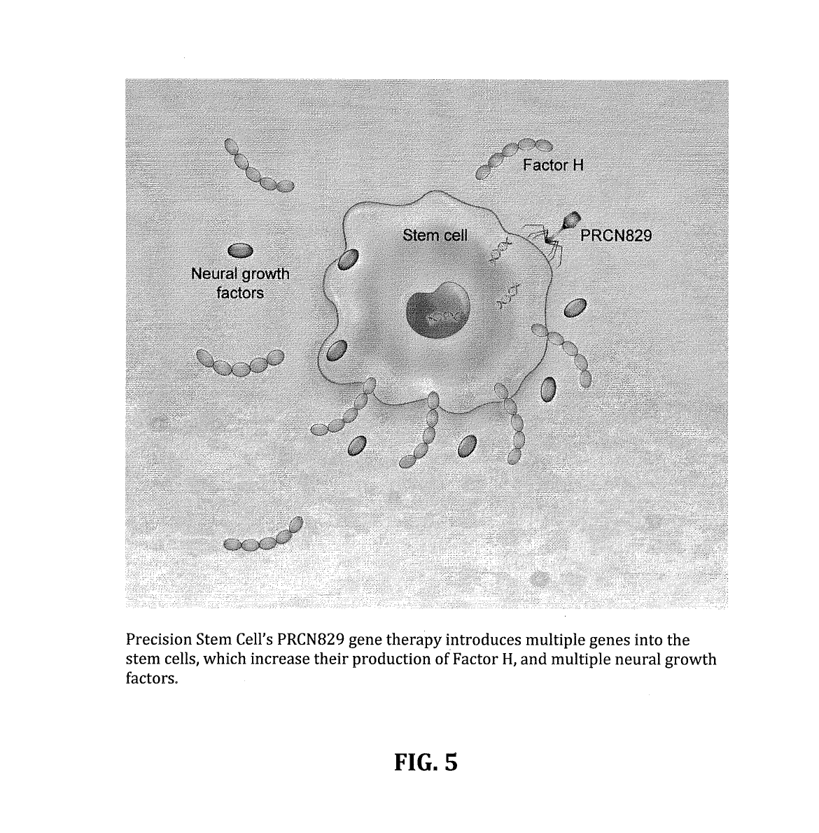

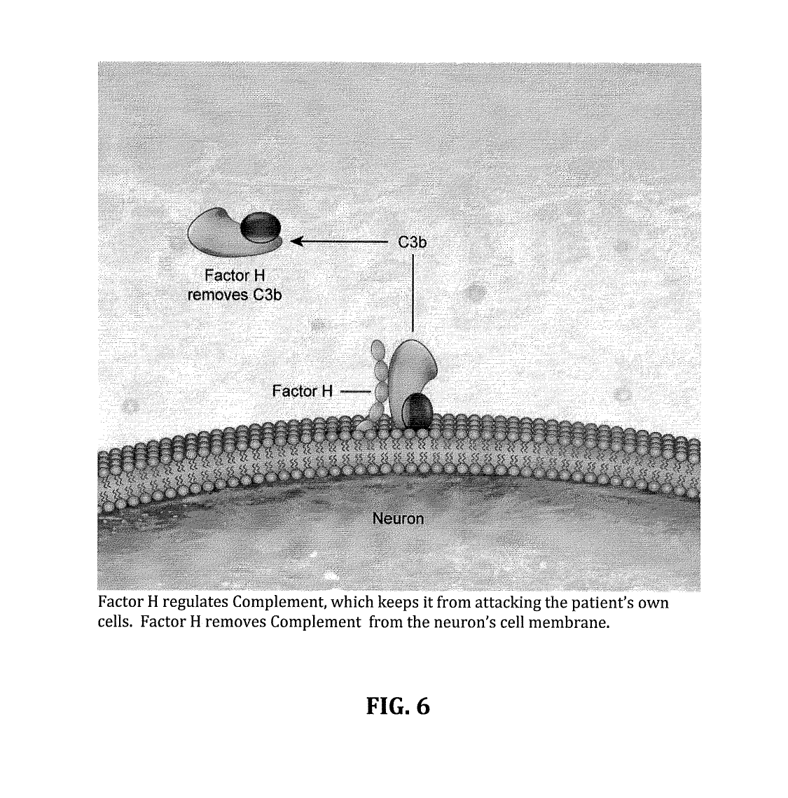

An example of the Complement process and treatment with gene therapy is shown in FIGS. 4-6. In FIG. 4 a gene therapy approach using Precision Stem Cell's PRCN 829 gene therapy system delivers multiple genes, which increase production of Factor H and multiple neural growth factors. This combination of genes inhibits the destruction of neurons in ALS. FIG. 5 illustrates the Precision Stem Cell's PRCN 829 gene therapy which introduces multiple genes into a stem cell, which then helps increase their production of Factor H, and multiple neural growth factors. FIG. 6 once again illustrates how Factor H regulates Complement, which keeps it from attacking the patient's own cells. Factor H removes Complement from the neuron's cell membrane.

Genetically engineering stem cells and some of the patients existing cells with Factor H will increase its production. This means that when the stem cells differentiate, they will continue to produce higher levels of Factor H. So, if they produce new nerves, those nerves will be protected from ALS and the attack of Complement. In addition, the gene therapy will transfer the gene for Factor H to the patient's normal existing cells. This will protect them as well.

To support this theory, there are several key factors: 1) ALS patients have high levels of Complement in the spinal fluid (CSF); 2) Drugs that inhibit Complement reduce symptoms of ALS and increase lifespan (ALS rodent models); 3) Mesenchymal stem cells produce Factor H, which inhibits Complement; 4) Patients see improvement or slowed progression when treated with Mesenchymal stem cells (results are short lived, just as Factor H production is as well); 5) Exposing stem cells to NSAIDS (non-steroidal anti-inflammatory drugs) inhibits production of Factor H; 6) Patients who took NSAIDS immediately after stem cell therapy saw reduced or no benefit (inhibited Factor H); and 7) Stem cells help repair joints by regeneration and inhibition of Complement. Patients begin having immediate relief in their pain, well before MRI evidence of cartilage regeneration. This is presumed due to Complement inhibition from Factor H.

Stem Cell Therapy Approach

An examplary stem cell therapy involves the use of adipose derived stem cells, which are then incubated with Selegeline, which results in preinduction into motor neurons. Initially, adipose tissue or fat is harvested via a minimally invasive liposuction. Then the fat is processed to separate the vascular stromal fraction of stem cells. A standard harvest and treatment was found to yield between 30-50 million stem cells. Once processed, the majority of stem cells are administered into the spine (spinal fluid) via a lumbar puncture. A small dilute fraction of cells are injected into a select muscle group. It is noted that the FDA currently does not allow the culture of stem cells and re-introduction at a later time in the United States, however other countries are not so rigorous, and it is believed that in the future the US will also allow for the less stringent standard as greater demonstration of the safety and the understanding of the techniques is further developed. The techniques allowing for culturing of stem cells and the reintroduction will help increase stem cell numbers. In addition, it will allow the storage of stem cells and enable the physician to perform multiple treatments from a single harvest. The current theory of stem cell therapy is that the stem cells can repair and regenerate motor neurons. In addition, the embodiments contemplated provide for stem cells that provide immune modulation, which further improves the disease state.

Utilization of Gene Therapy

Gene therapy has long been considered the future of medical therapy. Recent advancements have made this therapy a current reality. Gene therapy typically involves the insertion of desired genes into a cell. The genes are introduced into the cell via a vector, usually a virus. Currently, adeno-associated viruses (AAV) vectors seem to be the best option. These viruses can incorporate the gene into the cell and have a very good safety record in previously performed gene therapy.

Candidate Genes for Gene Therapy Approach for the Treatment of ALS

Embodiments of the present invention include numerous genes which are good for use in gene therapy based on their properties. The term “gene” as used herein refers to a nucleic acid molecule, such as a DNA molecule, a cDNA molecule, a gDNA molecule or RNA molecule that encodes a protein, which may or may not include regulatory sequences. Below is a list of non-limiting examples of genes contemplated for use in the present invention.

IGF-1: Insulin like growth factor 1(IGF-1) increased survival and delayed progression within the ALS mouse model.

TDP-43: TAR DNA binding protein 43 is a transcriptional repressor which has a complex association with neurodegenerative disorder. Mutated TDP-43 is shown to develop MND as well as under and over expression of wild type TDP-43. Contemplated gene therapy involves the knockdown of the mutant TDP-43 and increasing wild type expression when it is underexpressed.

EEAT2: Excitatory amino acid transporter 2 (EAAT2) is expressed in astrocytes and increases glutamate uptake which is neuroprotective to motor neurons. Increased expression of EAAT2 in the ALS mouse model delayed loss of motor neurons.

GDNF: Glial derived neurotrophic factor (GDNF) increased the number of neuromuscular connections and motor neuron cell bodies within the ALS mouse model.

Cardiotrophin-1: an IL-6 family cytokine which is neurotrophic for motor neurons. An ALS mouse model treated with AAV vector carrying Cardiotropin-1 gene had delayed neuromuscular degeneration and increased survival.

Brain-derived neurotrophic factor (BDNF): a protein which supports neuron survival and encourages growth of new neurons.

Ciliary neurotrophic factor (CNTF): a neurotrophic factor that is protective of neurons. Previous studies have shown that CNTF is protective to neurons that suffered damage, but the short half life (2.9 minutes) made administering it not feasible to administer it as a drug. The limitation of the use of CNTF as a drug can be overcome by the use of gene therapy.

Follistatin 344 (FSTN-344): an activin binding protein which results in increased muscle mass. The mechanism was thought to be from inhibition of myostatin, but there seems to be other mechanisms that are independent of myostatin. A study showed increased survival in the spinal muscular atrophy model (SMA). The follistatin may be beneficial in ALS patients by maintaining muscle mass. Studies on Russian dwarf hamsters treated intramuscularly with AAV-FSTN344 demonstrated an increase in life expectancy of 44%.

Factor H: a glycoprotein that is a regulator of complement. It inhibits complement activation against “self” proteins. Studies demonstrate that complement derangement may have a significant role in ALS. Studies have demonstrated that complement is activated against motor neurons and neuromuscular junctions in the SOD1 G93A ALS mouse model. Further demonstrations have shown that inhibition of complement with selective C5aR antagonist (PMX205) showed significant extension of survival and a reduction in end stage motor scores.

Further studies by the inventor have demonstrated success with the treatment of ALS patients using Selegeline reprogrammed adipose derived stem cells.

Therapeutic Targets

In ALS simultaneous treatment of the spinal cord (i.e., MN cell bodies and/or glial cells) and skeletal muscle (i.e., neuromuscular junctions [NKJs]) might be necessary to fully cover the pathways involved in MN degeneration.

Motor Neurons. Although MNs are known predominantly as the primary cell type implicated in the disease, increasing evidence indicates that they are perhaps not the sole target for therapeutic intervention in ALS. Gene therapy strategies for ALS had once focused mainly on treating MNs. However, defining a specific therapeutic target for ALS remains a challenge. Despite the selective vulnerability of MNs in ALS, astrocytes can play a modulatory yet detrimental role in the disease by triggering apoptotic and inflammatory mechanisms, thereby contributing to MN death. Moreover, reduced levels of glutamate transporters in astrocytes may cause impaired glutamate uptake and the consequent excitotoxicity occurring in ALS. Nonetheless halting MN degeneration is the ultimate goal of any therapeutic strategy for ALS.

Astrocytes. Down regulation of the excitatory amino acid transporter 2 (EEAT2). Expressed mainly in astrocytes, has been suggested as a cause of MN excitotoxicity. In fact, cells engineered to overexpress EAAT2 can dramatically increase glutamate uptake and confer neuroprotection on motor neurons in coculture systems in vitro. Increased expression of the EAAT2 in SOD1 mice can delay the loss of MNs in these double transgenic mice; conversely, a reduced amount of this receptor in SOD1 mice caused them to exhibit earlier MN loss. In conclusion, increasing the expression of glutamate receptors in glial cells could be beneficial for the treatment of ALS.

Neuromuscular junctions. Because end-plate denervation is one of the initial events in ALS, targeting NMJs at early stages can be critical to preserving MN connections. In new born SOD1 mice intramuscular injection of an adeno-associated viral vector encoding cardiotrophin-1 delayed neuromuscular degeneration. Similarly, in SOD1 rats ex vivo gene delivery of glial cell line-derived neurotrophic factor (GDNF) within muscles significantly increased the number of neuromuscular connections and, consequently, MN cell bodies during the midstages of the disease.

Example Treatment 1—Intra-spinal injection of reprogrammed adipose derived stem cells for improving the symptoms associated with ALS

Amyotrophic lateral sclerosis (ALS) is a severe progressive neurodegenerative disease with an unknown and poorly understood etiology. There are genetic and familial forms, but also environment and occupational exposure can result in risk factors for the development of ALS. Patients have a wide range of different clinical features. Inflammation and immune abnormalities have been detected in both human patients and the animal models. These immune abnormalities seem to be present regardless of the underlying cause. Embodied treatments have shown that intra-spinal injection of reprogrammed adipose derived stem cells results in some improvement of the symptoms of ALS. Patients treated with adipose derived ASC showed an early response, usually within the first few weeks of treatment. We postulate that this early response may be due to immune modulation. Embodiments effecting the alteration of immune response from adipose derived ASC may be utilized to better understand the disease process and better treatment options. Adipose derived ASC have shown positive effects in other disease processes, including autoimmune diseases and osteoarthritis. One common possible mechanism is the alteration or reduction of the complement component of the immune system. This supports embodiments wherein the modulation of complement C3 and C5 may play a key role in the treatment of ALS.

Mesenchymal stem cells have been demonstrated to produce a Complement regulating substance called Factor H (Tu, et al; Stem Cells and Development, Vol 19, Number 19, 2010). Factor H inhibits Complement activation, which inhibits that underlying attack of Complement on neurons in ALS.

Embodiments of the present invention have been used to treat 27 patients with ALS by intra-spinal (intra-thecal) and intra-muscular injection of adipose derived mesenchymal stem cells. A large number of these patients saw modest improvement of their symptoms. The patients improvement generally occurred within one month, and as soon as 1 day after treatment. Many of the patients seemed to experience a slower disease progression after treatment. Patients with aggressive or advanced disease seemed to have less noticeable benefits. In addition, we noted that patients who received non-steroidal anti-inflammatory drugs (NSAIDS) did not seem to experience any benefit. This further supports that Factor H, which is inhibited by NSAIDS, is involved in the improvement that ALS patients have from stem cell therapy.

Due to the fact that ALS patients see rapid improvement, this would reduce the likelihood that improvement is from nerve regeneration, which would take many months. The rapid improvement supports the concept that stem cells are producing substances, which inhibit the ALS disease process. The other fact that patients given NSAIDS, further supports that Factor H, at least to some degree, is that substance that inhibits the ALS process.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

What is claimed is:

1. A method of increasing the presence of Factor H in a mammalian subject, comprising:
a) harvesting adipose tissue from the subject;
b) purifying stem cells from the adipose tissue;
c) treating the stem cells with a compound that increases secretion of Factor H, optionally Selegeline; and
d) introducing the treated stem cells into the subject.

2. The method according to claim 1, where the increased factor H secretion results in complement inhibition.

3. A method of treating a motor neuron disease comprising:
a) harvesting stem cells from a patient with the motor neuron disease;
b) genetically altering the stem cells by the addition of one or more genes selected from the group consisting of IGF-1, TDP-42, and factor H;
c) administering the genetically altered stem cells systemically to the patient;
wherein the systemic administration serves to carry added genes which are protective against motor neuron death, and which further increases the amount of selective genes which further reduce the effects of motor neuron disease; and
d) optionally administering additional selected gene therapy components intramuscularly, wherein the IM administration serves to protect the axon and assist with maintaining muscle mass and function.
